(19) United States
(12) Patent Application Publication
(10) Pub. No.: US 2006/0094949 A1
(43) Pub. Date: May 4, 2006

(54) Methods and systems for real time response to medical emergencies

(76) Inventors: Charles Kevin Coonce, Parma, MI (US); Mark James Maloney, Jackson, MI (US)

Correspondence Address: ICE MILLER LLP, ONE AMERICAN SQUARE, SUITE 3100, INDIANAPOLIS, IN, 46282-0200, US

(21) Appl. No.: 10/976,569

(22) Filed: Oct. 29, 2004

(51) Int. Cl. A61B 5/05

(52) U.S. Cl. 600/407

(57) ABSTRACT

An emergency medical system, computer readable mediums with a computer programs for and methods for responding to an emergency in real time are disclosed. The emergency medical systems includes a emergency medical station and an operations center. The emergency medical station includes a controller, a video camera, a communication module, a display screen and a medical device compartment with a locking mechanism. Real time video data is transmitted from the emergency medical station to the operations center and real time video data is received from the operations center at the emergency medical station. A determination is made regarding whether the medical device compartment should be unlocked based on the real time two-way video data exchange between the emergency medical station and the operations center. The locking mechanism of the medical device compartment is unlocked based on the determination. In non-emergency situations, user interest data may be shown on the display screen for viewing. Users may interact with some forms of user interest data via a user interface.

### FIELD OF THE INVENTION

The present invention relates generally to methods and systems for responding to medical emergencies. In particular, the invention relates to a method and system for providing a two way real time video communication link between an emergency medical station at the site of an emergency and a medical professional at an operations center.

### BACKGROUND OF THE INVENTION

Medical emergencies often occur in public areas, such as for example, schools, offices, stores and other public areas. Many medical emergencies require prompt medical attention to maximize the chances of survival. Many businesses and building managers have invested large sums of money to provide public access to potentially life saving emergency medical devices that can be used by a bystander if a paramedic and/or other trained professional are not immediately available to administer necessary medical assistance. At present, the businesses and other entities bear the total cost of providing availability and access to potentially life saving medical devices.

For example, heart disease is the leading cause of death in the United States, with nearly half a million people dying of cardiac arrest every year. Individuals suffering from cardiac arrest can often be successfully treated with the proper use of an external defibrillation by a bystander. However, improperly applying external defibrillation or applying unnecessary external defibrillation to an individual may aggravate that person's medical condition.

U.S. Pat. No. 5,593,426 and related patent, U.S. Pat. No. 5,728,878, disclose a system with a plurality of external defibrillator systems in communication with a remote medical response facility. In the event of an emergency, a user establishes a real time two way audio communication link between the defibrillator system and the medical response facility. The medical response facility provides the user with access to the defibrillator by transmitting a return signal to the defibrillator system. The defibrillator system includes a display screen that is configured to display text, still picture or video instructions transmitted from the medical response facility. However, the defibrillator system is not equipped to communicate video data from the user to the medical response facility thereby limiting a medical expert's ability to properly assess a medical emergency. Furthermore, inability to monitor the user's actions and visually assess a patient's medical condition may hinder a medical expert's ability to effectively guide the user through necessary emergency procedures. At present, the businesses and managers bear the total cost of providing such devices.

U.S. patent application Ser. No. 10/335,542 discloses an external defibrillator system that automatically establishes a communication link between the external defibrillator system and a safety agency in response to the actuation of a button by a user. The communication link supports two way real time audio communication between the external defibrillator system and one way real time video communication from the safety agency to the external defibrillator system. The safety agency personnel provide audio and visual instructions to guide a user through defibrillator operation. However, the external defibrillator system is not equipped to communicate video data from the user to the safety agency. The lack of visual feedback of the user's actions and the patient's medical condition limits safety agency personnel's ability to assess the patient's condition and the effectiveness of the user's actions.

Prior art medical emergency systems are typically sought by potential users only in emergency situations. In many cases, potential users pay little attention to the location and nature of the medical emergency devices on a day-to-day basis. As a result, when faced with an emergency situation, potential users may fail to recall the location, the availability and the nature of such medical emergency systems thereby losing valuable time in properly assisting a person in distress.

It is desirable, therefore, to provide systems and methods for responding to a medical emergency situation that overcomes one or more of the prior art limitations described above.

### SUMMARY OF THE INVENTION

One aspect of the present invention provides an emergency medical station including a controller, a video camera, a communication module, a display screen and a medical device compartment with a locking mechanism. The controller is communicatively coupled to the video camera, the communication module, the display screen and the locking mechanism. The communication module is operable to transmit real time video data from the video camera to an operations center, to receive real time video data from the operations center and to receive a release signal from the operations center. The display screen is operable to display the received real time video data. The locking mechanism is operable to unlock the medical device compartment responsive to the release signal.

Another aspect of the invention provides a method of responding to an emergency situation. The method includes establishing a communication link between an emergency medical station and an operations center, transmitting real time video data from the emergency medical station to the operations center, receiving real time video data from the operations center at the emergency medical station, determining whether a medical device compartment having a locking mechanism should be unlocked based on the real time video data exchange between the emergency medical station and the operations center; and unlocking the locking mechanism based on the determination.

Another aspect of the invention provides a computer readable medium storing a computer program for responding to an emergency situation. The computer program includes computer readable code for establishing a communication link between an emergency medical station and an operations center, computer readable code for transmitting real time video data from the emergency medical station to the operations center, computer readable code for receiving real time video data from the operations center at the emergency medical station, and computer readable code unlocking a locking mechanism of a medical device compartment responsive to a release signal received from the operations center wherein, the release signal is generated from the operations center based on an assessment of the real time video data exchanged between emergency medical station and the operations center.

Another aspect of the invention provides an emergency medical station. The emergency medical station includes a controller communicatively coupled to a video camera, a communication module and a display screen. The controller is operable to place the emergency medical station in one of a non-emergency mode and an emergency mode. The communication module is operable to transmit real time video data from the video camera to an operations center in emergency mode and to receive real time video data from the operations center in emergency mode. The display screen is operable to display user interest data in non-emergency mode and operable to display the real time video data received from the operations center in emergency mode.

Another aspect of the invention provides a method for responding to an emergency situation. The method includes providing an emergency medical station having a default non-emergency mode wherein user interest data is displayed on a display screen in non-emergency mode, placing the emergency medical station in emergency mode responsive to activation of an emergency switch, transmitting real time video data from the emergency medical station to the operations center in emergency mode, receiving real time video data from an operations center at the emergency medical station in emergency mode, and displaying the real time video received from the operations center on the display screen in emergency mode.

Another aspect of the invention provides a computer readable medium storing a computer program. The computer program includes computer readable code for placing an emergency medical station in a default non-emergency mode and for placing the emergency medical station in emergency mode responsive to an emergency signal, computer readable code for displaying user interest data on a display screen in non-emergency mode, computer readable code for transmitting real time video data from the emergency medical station to the operations center in emergency mode, computer readable code for receiving real time video data from the operations center at the emergency medical station in emergency mode, and computer readable code for displaying the real time video data received from the operations center on the display screen in emergency mode.

Another aspect of the invention provides an emergency medical station. The emergency medical station includes a display for displaying user interest data in non-emergency mode. The display of user interest data draws the attention of a potential user to the location of the emergency medical station in a welcome manner.

Another aspect of the invention provides a method for finding the placement of an emergency medical station in a first party facility. The method includes the steps receiving a request for placement of an emergency medical station at the first party facility from a first party, placing the emergency medical station in the first party facility, displaying user interest data associated with a second party on an emergency medical station display, receiving compensation from the second party for displaying the user interest data associated with the second party, funding the costs associated with the placement of the emergency medical station in the first party facility using the compensation received from the second party.

Anther aspect of the invention provides an emergency response system includes an operations center communicatively coupled to a plurality of emergency medical stations. The operations center is staffed around the clock with medical experts available to respond a an emergency signal received from one of the plurality of medical emergency stations. An emergency signal is generated upon activation of an emergency button on a first of the plurality of emergency medical stations. A real time two way video communication link is established between the first emergency medical station and the operations center. A medical expert at the operations center guides the user of the emergency medical station through a medical procedure.

### BRIEF DESCRIPTION OF THE DRAWINGS

The present invention is illustrated by way of example and not limited in scope to the accompanying figures, in which like references indicate similar elements, and in which:

FIG. 1 is a schematic block diagram of an embodiment of a real time emergency response system in accordance with the principles of the present invention;

FIG. 2 is a schematic block diagram of an embodiment of a content distribution center in accordance with the principles of the present invention;

FIG. 3 is a schematic block diagram of an embodiment of an operations center in accordance with the principles of the present invention;

FIG. 4 is a schematic block diagram of an embodiment of an emergency medical station in accordance with the principles of the present invention;

FIG. 5 is an illustration of an embodiment of an emergency medical station in accordance with the principles of the present invention;

FIG. 6 is a flowchart of one embodiment of a method for placing the emergency medical station in the appropriate operating mode in accordance with the principles of the present invention;

FIG. 7a-b is a flowchart of one embodiment of a method of operating a emergency medical station in non-emergency mode in accordance with the principles of the present invention; and

FIG. 8a-c is a flowchart illustrating one embodiment of a method of responding to an emergency situation in real time in accordance with the principles of the present invention.

### DETAILED DESCRIPTION OF THE PREFERRED EMBODIMENTS

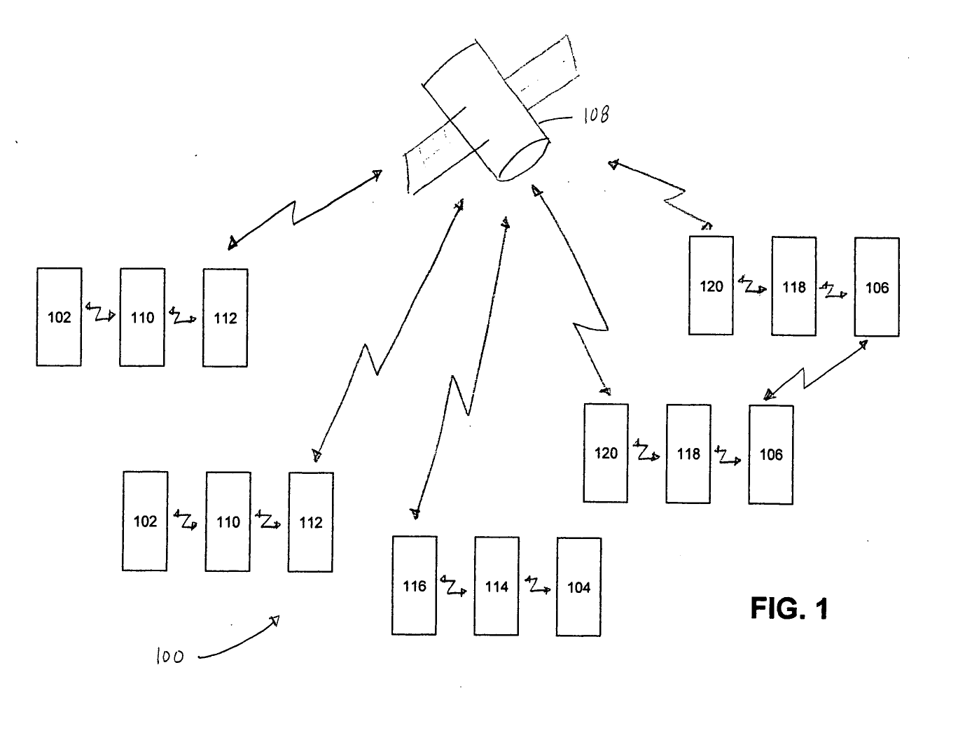

FIG. 1 is a schematic diagram of one presently preferred embodiment of a real time emergency response system 100, in accordance with the principles of the present invention. The real time emergency response system 100 includes one or more emergency medical stations 102, one or more content distribution centers 104 and one or more operations centers 106.

In one presently prefered embodiment, each of the emergency medical stations 102 can be placed in one of two operating modes, a non-emergency mode and an emergency mode, with the non-emergency mode being the default operating mode. The content distribution centers 104 create and transmit user interest data for display on individual emergency medical stations 102 in non-emergency mode. The emergency medical station 102 stores the received user interest data. The user interest data selected by a content distribution center 104 for display on a particular emergency medical station 102 may be dependent on the location of that emergency medical station 102 and on the specific requests of the entity authorizing the placement of the emergency medical station 102. For example, an emergency medical station 102 located in a dentist's office may display user interest data directed to dental needs. The display of user interest data may unobtrusively and/or attractively draw the attention of potential users of the emergency medical station 102 to the location, availability and nature of the emergency medical station 102 in a non-emergency environment. This increases the probability that a potential user will recall the location and availability of the medical emergency station 102 when faced with an emergency situation.

In the event of an emergency, a user can activate an emergency button and place the emergency medical station 102 in emergency mode. The emergency medical station 102 issues an emergency response request to the operations center 106. A real time two-way audio/video communication link is immediately established between the emergency medical station 102 and an operations center 106. The operations center 106 includes a plurality of medical expert stations where each medical expert station is staffed by a medical expert. The operations center 106 is staffed by medical experts around the clock to provide medical emergency guidance as needed in response to placement of one or more emergency medical stations 102 in emergency mode. The medical experts are medical professionals, such as for example, paramedics. More preferably, a real time two way audio/video communication link is created between the emergency medical station 102 and an available medical expert station. The medical expert at the medical expert station guides the user through the medical procedures necessary to deal with the emergency situation. The emergency medical station 102 is also used to store a medical device in a locked medical device compartment. The medical expert grants the user access to the medical device if the medical expert determines that use of the medical device is warranted.

In one presently preferred embodiment of the real time emergency response system 100, the one or more emergency medical stations 102 are communicatively coupled to the one or more content distribution centers 104 and the one or more operations centers 106 via a satellite communication system.

Each emergency medical station 102 is configured to transmit and receive audio and video data to and from a communication satellite 108 via one or more routers 110 and a satellite dish 112. The emergency medical station 102 may be communicatively coupled to the router 110 via a wired communication link, a wireless communication link or a combination wired and wireless communication link. The router 110 may be communicatively coupled to the satellite dish 112 via a wired communication link, a wireless communication link or a combination wired and wireless communication link.

The content distribution center 104 is configured to transmit and receive audio and video data to and from a communication satellite 108 via one or more routers 114 and a satellite dish 116. Each content distribution center 104 may be communicatively coupled to the router 114 via a wired communication link, a wireless communication link or a combination wired and wireless communication link. The router 114 may be communicatively coupled to the satellite dish 116 via a wired communication link, a wireless communication link or a combination wired and wireless communication link.

Each medical expert station at an operations center 106 is configured to transmit and receive audio and video data to and from a communication satellite 108 via one or more routers 118 and a satellite dish 120. Each medical expert station may be communicatively coupled to the router 118 via a wired communication link, a wireless communication link or a combination wired and wireless communication link. The router 118 may be communicatively coupled to the satellite dish 120 via a wired communication link, a wireless communication link or a combination wired and wireless communication link.

It should be noted that while the emergency medical stations 102 have been described as preferably communicatively coupled to the one or more content distribution centers 104 and the one or more operations centers 106 via a satellite communication system, other forms of communication networks that enable high speed audio and video communication are also considered to be within the scope of the invention. Examples of such communication systems include, but are not limited to, T1 line, T3 line, OC3 fiber, OC12 fiber, OC48 fiber, OC192 fiber, digital subscriber line (DSL) or any other high speed communication lines that may be apparent to one having ordinary skill in the art. A combination of wired and wireless communications networks may also be employed without departing from the spirit of the invention.

Each of the operations centers 106 are communicatively coupled to the other operations centers 106′. In the event an operations center 106 receives too many emergency response requests at the same time, the operations center 106 can hand off one or more of the received emergency response requests to a second operations center 106′ that has the available capacity to handle the request. All of the audio and video data received from the emergency medical station 102 issuing the emergency response request is routed from the first operations center 106 to the second operations center 106′.

The operations centers 106, 106′ maybe communicatively linked via high speed communication links, such as for example, T1 line, T3 line, OC3 fiber, OC12 fiber, OC48 fiber, OC192 fiber, DSL line or any other high speed communication lines that may be apparent to one having ordinary skill in the art. The operations centers 106, 106′ may also be communicatively linked via a wireless communication network, such as for example, a satellite communication network. A combination of wired and wireless communication networks may also be employed to create communication links between the operation centers 106, 106′.

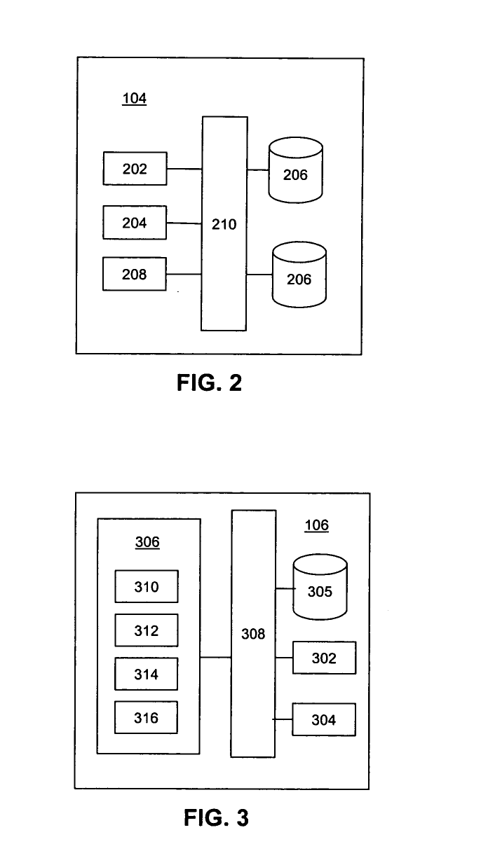

Referring to FIG. 2, a schematic block diagram of another preferred embodiment of a content distribution center 104 in accordance with the principles of the present invention is shown. The content distribution center 104 includes one or more data modems 202, one or more content distribution servers 204, one or more databases 206 and one or more content distribution stations 208 communicatively coupled via a network system 210.

The content distribution center 104 is staffed with one or more content distribution advisors. The content distribution advisors create and update the user interest data, and coordinate the distribution of selected user interest data to individual emergency medical stations 102 via the content distribution stations 208. All of the user interest data is stored in the databases 206. The user interest data selected by a content distribution advisor for transmission to a particular emergency medical station 102 may depend on the location of that emergency medical station 102 and on specific requests made by the entity authorizing the placement of the emergency medical station 102. When a content distribution advisor authorizes the transmission of selected user interest data to an emergency medical station 102 via a content distribution station 208, a content distributions server 204 retrieves the selected user interest data from a database 206. The retrieved user interest data is forwarded to a data modem 202 for transmission to the designated emergency medical station 102 via the satellite communication system.

In one preferred embodiment, the user interest data may include product data. Product manufactures and product retailers wishing to advertise their product, work with a content distribution advisor to coordinate the creation and display of their product data at one or more designated medical emergency stations 102. Examples of product data include, but are not limited to, product advertisement data and product promotional data. Product promotional data may include, but is not limited to, health care tips associated with particular products, coupons and rebates.

In another embodiment, the user interest data may include displays of forms configured to accept user supplied data via a user interface at the emergency medical station 102. Such forms include, but are not limited to, sweepstakes entry forms, market survey forms, or requests for additional information regarding an advertised product. The user can provide the information requested in the forms via the user interface. In one embodiment, prompts are provided to the user to ensure that the requested information is supplied.

In cases where the user requests additional information regarding an advertised product, the user supplied data includes user contact data. Examples of user contact data include, but are not limited to, user name, user address, user phone number, and user email address. The user contact data is initially stored in the emergency medical station 102 and then forwarded to the content distribution center 104 responsive to a data request from the content distribution center 104. The content distribution center 104 forwards the user contact data to the appropriate product retailer or manufacturer so that they can directly provide the user with the requested product data.

In another preferred embodiment, the user interest data may be configured in view of the area where the emergency medical station 102 is being placed. For example, if the medical emergency station 102 is located in a school, the user interest data may include school related data, such as for example class schedule information.

In one embodiment, the entity responsible for authorizing the placement of one or more emergency medical stations 102 may be granted limited access to the database 206 to modify and/or update the user interest data displayed on those emergency medical stations 102.

Referring to FIG. 3, a schematic block diagram of another preferrred embodiment of an operations center 106 in accordance with the principles of the present invention is shown. The operations center 106 includes a operations controller 302, one or more data modems 304, one or more operations databases 305, and one or more medical expert stations 306 communicatively coupled via a network system 308. Each medical expert station 306 is equipped with a video camera 310, a display screen 312, a microphone 314 and one or more speakers 316.

In the event of an emergency, such as for example, the user sees a person in medical distress, a user activates an emergency button thereby placing the emergency medical station 102 in emergency mode. The emergency medical station 102 issues an emergency response request to the operations center 106. A communication link is immediately established between the emergency medical station 102 and an operations center 106. The operations controller 302 assigns an available medical expert station 306 to the emergency medical station 102 and immediately establishes a real time two-way audio/video communication link between the emergency medical station 102 and the assigned medical expert station 306. The real time video data received from the emergency medical station 102 is displayed on the display screen 312. The real time audio data received from the emergency medical station 102 is transmitted via the speakers 316. The real time video of the live medical expert is recorded by the video camera 310 and transmitted to the emergency medical station 102. The real time audio data of the live medical expert is picked up by the microphone 314 and transmitted to the emergency medical station 102.

The operations controller 302 forwards the real time audio/video data from the medical expert station 306 to the data modem 304 for transmission to the emergency medical station 102 via the satellite communication system. The real time two-way audio/video communications between the medical expert at the medical expert station and the user at the emergency medical station 102 are recorded and stored in the operations database 305.

The medical expert at the medical expert station 306 informs a local emergency response facility of the emergency situation and provides real time instructions to the user at the emergency medical station 102. Based on the real time two-way the audio/video communication with the user at the emergency medical station 102, the live medical expert determines whether use of the medical device stored at the emergency medical station 102 is necessary. If the medical expert determines that use of the stored medical device is warranted, the medical expert authorizes transmission of a release signal from the operations center 106 to the emergency medical station 102 to release the medical device compartment door locking mechanism 140. The release signal is transmitted from the operations center 106 to the emergency medical station 102 via the satellite communication system. The medical expert instructs the user on proper use of the medical device. If the medical expert determines that use of the stored medical is unnecessary, the medical expert continues to guide the user through other emergency procedures in an attempt to assist the person in medical distress until local emergency response facility personnel arrive and take charge of the emergency situation.

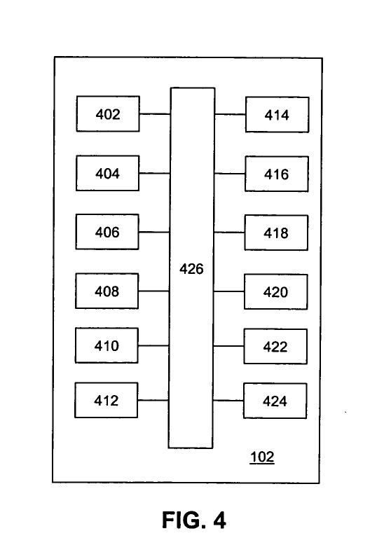

Referring to FIG. 4, a schematic block diagram of another preferred embodiment of an emergency medical station 102 in accordance with the principles of the present invention is shown. The emergency medical station 102 includes a controller 402 that is communicatively coupled to a local memory 404, a communication module 406, a display screen 408, a video camera 410, a microphone 412, one or more speakers 414, a printer 416, an alarm system 418, an emergency switch 420, a locking mechanism 422, and a door tamper alarm 424 via a network 426. In one embodiment, the emergency medical station 102 includes a global positioning (GPS) unit.

In one embodiment, the controller 402 is a digital signal processor (DSP). In another embodiment processor may be implemented as a microcontroller, microprocessor, controller, host processor, or communications processor. The processor may be implemented as an application specific integrated circuit (ASIC). In another embodiment, the processor may be implemented as a processor working in conjunction with a central processing unit (CPU) performing the function of a general purpose processor. The processor executes various computer programs that control programming and operational modes of electronic and mechanical systems within the emergency medical station 102, and communications via the communication module 406.

The local memory 404 is used to store user interest data received from the content distribution center 104, user contact data, and audio/video communications between a user at the emergency medical station 102 and a medical expert at the operations center 106 when the emergency medical station 102 is in emergency mode. In one embodiment, the emergency medical station specific location data is stored in the local memory 404.

The communication module 406 includes a transmitter and a receiver. The transmitter transmits the real time video data recorded by the video camera 410 and the real time audio data picked up by the microphone 412 from the emergency medical station 102 to the operations center 106 when the emergency medical station 102 is in emergency mode. The transmitter also transmits stored user contact data to the content distribution center 104 responsive to a data request received at the emergency medical station 102 from the operations center 106. The receiver receives user interest data from the content distribution center 104 and real time audio/video data from the operations center 106.

In one embodiment the display screen 408 is a touch screen display. In another embodiment, the display screen 408 is a cathode ray tube (CRT) display. In another embodiment, the display screen 408 is a liquid crystal (LCD) display. While a number of types of display screens 408 have been described other types of display screens 408 are also considered to be within the scope of the invention.

Also while a touch screen display has been described as a preferred user interface, other types of user interfaces, such as for example, a pointer control for manipulating a pointer on the display screen 408 to input user selections, a keyboard, a bar code reader, and a card reader are also considered to be within the scope of the invention. In one embodiment, the printer 416 consists of a thermal printer. In another embodiment, the printer 416 is a dot matrix printer. In another embodiment, the printer 416 is an ink jet printer. Other types of printers may be used without departing from the spirit of the invention. A user may choose to print selected user interest data. The selected user interest data is printed on the printer 416.

In one embodiment, the emergency switch 420 consists of a push button. In another embodiment, the emergency switch 420 consists of a lighted push button. In another embodiment, the emergency switch consists of a lighted red push button. The emergency switch 420 generates an emergency signal when activated. While a number of types of emergency switches 420 have been described, it should be noted that the use of other colors and forms of emergency switches 420 are also considered to be within the scope of the invention. The controller 402 places the emergency medical station 102 in emergency mode responsive to the emergency signal.

The alarm system 418 is activated responsive to the activation of the emergency switch 420. The controller 402 coordinates the activation of the alarm system 418. In one embodiment, the alarm system 418 includes a siren. In another embodiment, the alarm system 418 includes a strobe light. In yet another embodiment, the alarm system 418 consists of a combination of a siren and a strobe light. While a number of different alarm systems 418 have been described, other types of alarm systems 418 are also considered to be within the scope of the invention. In one embodiment, the alarm system 418 is activated for a pre-defined period of time following activation of the emergency switch 420. In one embodiment, the pre-defined period of time is approximately ten seconds.

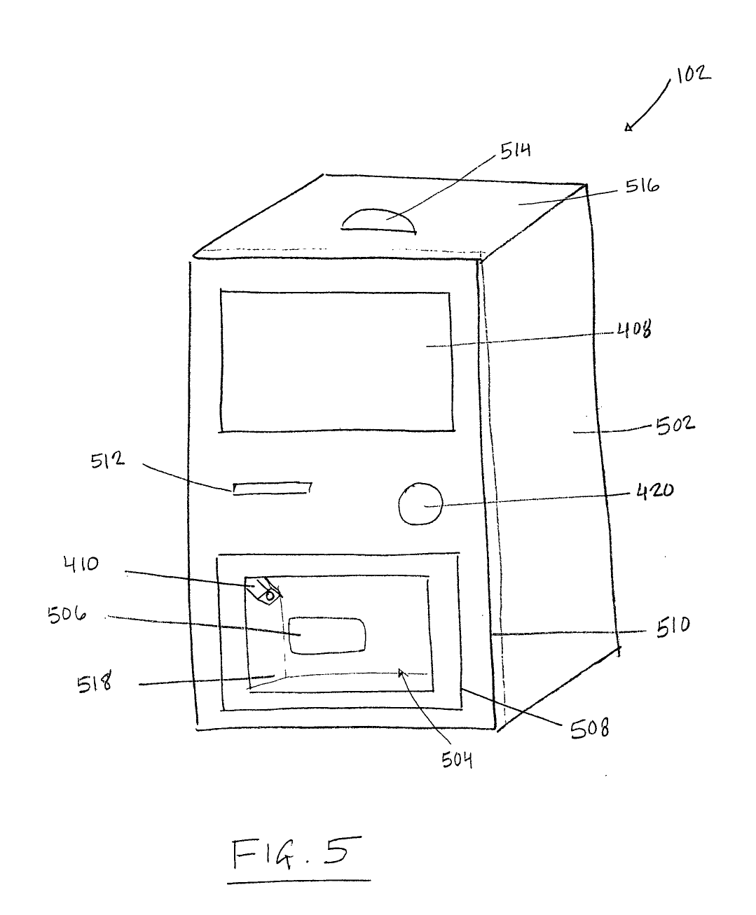

FIG. 5 shows another preferred embodiment of an emergency medical station 102 in accordance with the principles of the present invention is shown. Referring to FIG. 4 and FIG. 5, the emergency medical station 102 is integrated into a cabinet 502. The cabinet 502 includes two separate compartments an upper compartment and a medical device compartment 504. The medical device compartment 504 used to store a medical device 506. The medical device 506 can be accessed by opening a compartment door 508. The compartment door 508 is locked using the locking mechanism 422. The locking mechanism 422 is controlled by the controller 402 and is maintained in a default locked position.

In the event of an emergency situation, the locking mechanism 422 is unlocked responsive to a release signal received from the operations center 106 thereby permitting a user to access the medical device 506. In one embodiment, the locking mechanism 422 is a servo driven locking mechanism. In one embodiment, the locking mechanism 422 is unlocked responsive to a disruption in power supplied to the emergency medical station 102. In another embodiment, the locking mechanism 422 is released after a pre-defined period of time following a failure to establish a real time communication link between the emergency medical station 102 and the operations center 106 upon activation of the emergency switch 420. In one embodiment, the pre-defined period of time is approximately 10 seconds. In one embodiment, the medical device compartment 504 includes a door tamper alarm 424 that is activated if the compartment door 512 is forced open. In one embodiment, the door tamper alarm 424 includes a siren.

The cabinet 502 including a cabinet door 510. The cabinet door 510 provides access to both the upper compartment and the medical device compartment 504. The emergency medical station circuitry, including, the controller 120, local memory 122, the communication module 124, and the printer 416 are disposed within the upper compartment behind the display screen 408.

The display screen 408 is held in place within an opening in the upper section of the cabinet door 510. A printer slot 512 disposed below the display screen 408 and extends through the cabinet door 510. The printed material is guided from the printer 416 disposed within the upper compartment inside the cabinet 502 through the printer slot 512.

The emergency switch 420 is a red lighted push button and is affixed to the cabinet door 510 next to the printer slot 512 and below the display screen 408. The alarm system 418 includes a red strobe light 508 and a siren. The red strobe light 508 is disposed on an upper surface 516 of the cabinet 502.

As mentioned previously, the lower compartment is the medical device compartment 504. The compartment door 508 includes a transparent window 518. In one embodiment, the transparent window 518 is a Plexiglas window. The medical device 506 is stored within the medical device compartment 504 and is visible through the transparent window 518. In one embodiment, the medical device 506 consists of a standalone portable automatic defibrillator device. In one embodiment, the video camera 410 is disposed within the medical device compartment 504 behind the transparent window 518.

In one embodiment, the emergency medical station 102 includes a back up power supply (not shown). The back up power supply is activated responsive to a failure of the primary power supply powering the emergency medical station 102. In one embodiment, the back up power supply has the capacity to power the emergency medical station 102 for approximately an hour.

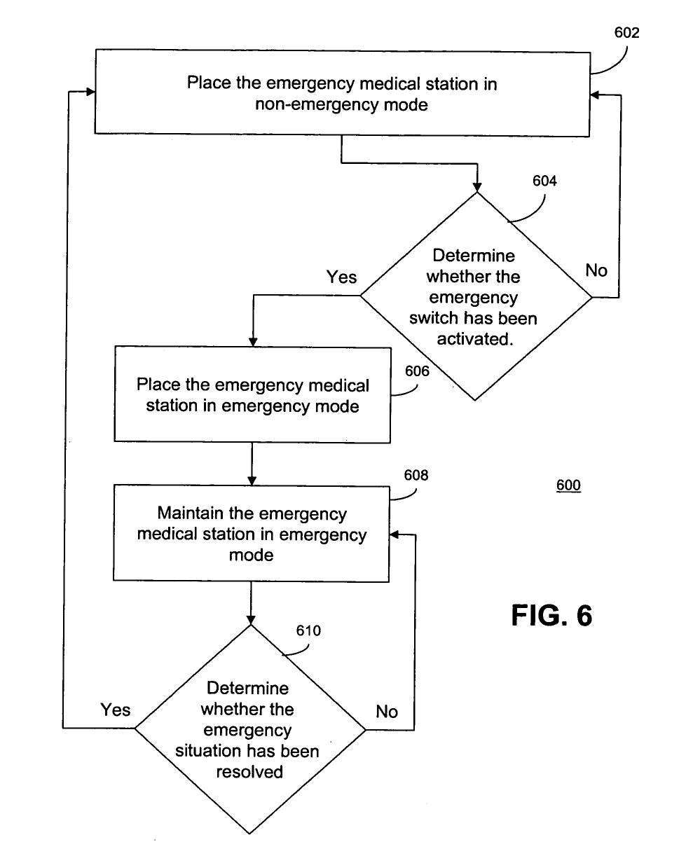

Referring to FIG. 6, a flowchart illustrating a presently preferred embodiment of a method for placing the emergency medical station in the appropriate operating mode 600 in accordance with the principles of the present invention is shown. Each medical emergency station 102 can be individually placed in one of two operating modes: a non-emergency mode and an emergency mode. The non-emergency mode is the default operating mode for the emergency medical station 102. The medical emergency station 102 is placed in emergency mode when a user activates the emergency switch 420. Typically a user activates the emergency switch 420 when the user sees a person in medical distress.

The method 600 begins at step 602 with placing the emergency medical station 102 in non-emergency mode. The emergency medical station 102 is placed in non-emergency mode when powered on for operation. The non-emergency mode is the default operating mode for the emergency medical station 102. The controller 402 displays user interest data on the display screen 408 in non-emergency mode.

The controller 402 is communicatively coupled to the emergency switch 420. At step 604, the controller 402 determines whether the emergency switch 420 has been activated by a user. If the emergency switch 420 has not been activated by a user, the emergency medical station 102 continues to operate in non-emergency mode as indicated in step 602. If the emergency switch 420 has been activated, the emergency switch 420 responsively generates an emergency signal. The controller 402 places the emergency medical station 102 in emergency mode responsive to the emergency signal at step 606.

In emergency mode, the emergency medical station 102 establishes a real time two-way audio/video communication link with the operations center 106. A medical expert at the operations center 106 communicates with the user and guides the user through medical procedures that the medical expert deems appropriate to assist the medically distressed person or patient. The medical expert also contacts a local emergency response facility, such as for example a local hospital, a local fire department or a local police department, and apprises them of the location and nature of the emergency situation.

At step 608, the emergency medical station 102 is maintained in emergency mode until the emergency situation is resolved. The emergency situation is considered to be resolved after local emergency personnel arrive at the scene of the emergency and the medical expert updates the emergency personnel regarding the patient's medical condition, the medical procedures performed on the patient and the patient's response to the medical procedures. At step 610, the controller 402 determines whether the emergency situation has been resolved. The medical expert at the operations center 106 issues an emergency resolved signal to the emergency medical station 102 upon resolution of the emergency situation. If the controller 402 does not receive a emergency resolved signal, the controller 402 continues to maintain the emergency medical station in emergency mode as indicated in step 608. If the controller 402 receives the emergency resolved signal, the controller 402 returns the emergency medication station 102 to non-emergency mode as indicated in step 602. The controller 402 resumes displaying user interest data on the display screen.

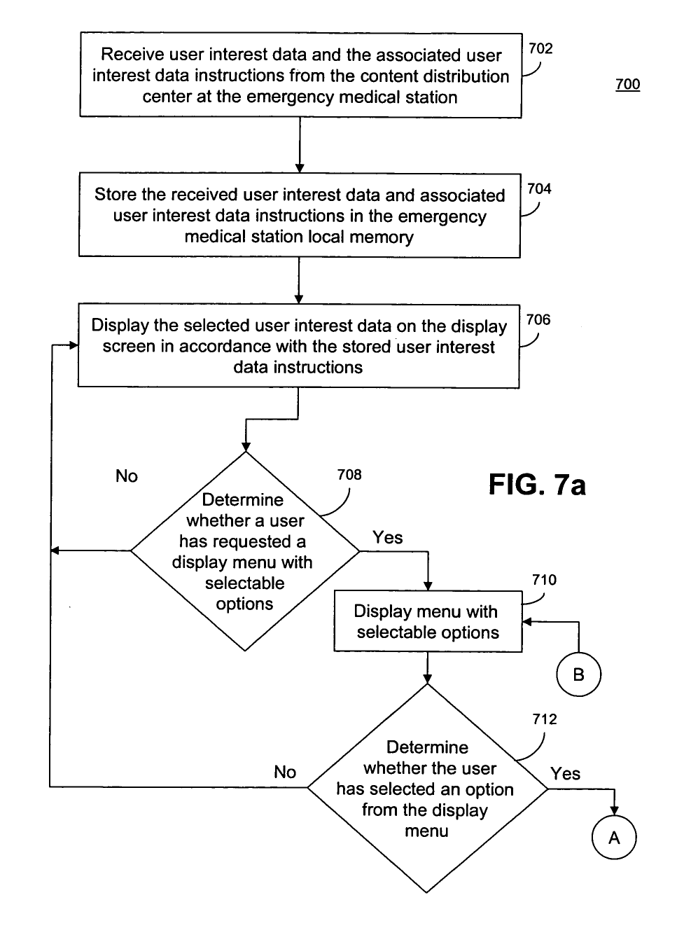

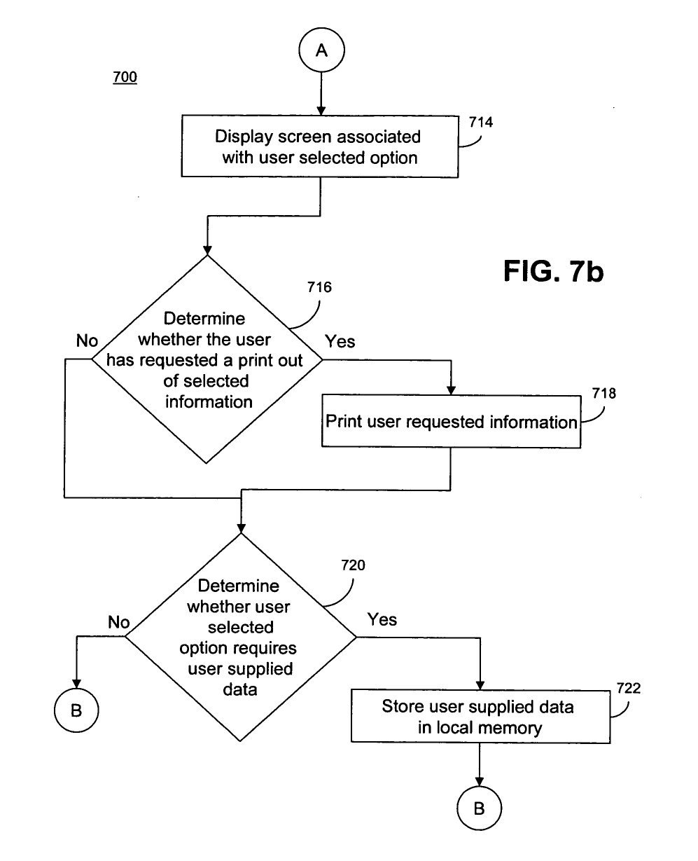

Referring to FIG. 7a-b, a flowchart illustrating a presently preferred embodiment of a method of operating the emergency medical station in non-emergency mode 700 in accordance with the principles of the present invention is shown. As mentioned previously, the emergency medical station 102 is placed in non-emergency mode upon being powered on. The non-emergency mode is the default operating mode.

The method 700 begins at step 702 with the emergency medical station 102 receiving user interest data and user interest data instructions from the content distribution center 104. The content distribution advisor identifies the specific user interest data for each individual emergency medical station 102. The user data instructions provide the controller 402 with instructions for displaying the user interest data on the display screen 408 at the emergency medical station 102. In one embodiment, the user interest data instructions define the sequence for displaying selected user interest data on the display screen 408.

The user interest data and the user interest data instructions are transmitted from the content distribution center 104 to the emergency medical station 102 via the satellite communication system. More specifically, the content distribution advisor identifies and flags the specific user interest data and associated user interest data instructions for the emergency medical station 102 at the content distribution station 208. The content distribution server 116 retrieves the flagged user interest data from the database 206 and routes the retrieved user interest data and the associated user interest data instructions to data modem 202. The data modem 202 forwards the user interest data and the associated user interest data instructions to the communication satellite 108 via the router 114 and the satellite dish 116.

The communication satellite 108 transmits the user interest data and the associated user interest data instructions to the destination emergency medical station 102 via the satellite dish 112 and the router 110. The communication module 406 receives the user interest data and the associated user interest data instructions. At step 704, the controller 402 stores the user interest data and the associated user interest data instructions in the local memory 404. At step 706, the controller 402 displays selected user interest data on the display screen 408 in accordance with the stored user interest data instructions.

In another embodiment, the user interest data and the associated user interest data instructions are maintained at the content distribution center 104. A constant communication link is maintained between the emergency medical station 102 and the content distribution center 104. The user interest data is transmitted to the emergency medical station 102 for display on the display screen 408 in accordance with the user interest data instructions. In this embodiment, the user interest data and the associated user interest data are never stored in the local memory 404.

In one embodiment, the display screen 408 is a touch screen display screen. The touch screen display screen is a user interface. While the controller 402 is displaying specific user interest data in accordance with user interest data instructions on the display screen 408, the user has the option of interrupting the display sequence by requesting that a display menu be displayed on the display screen 408 via the touch screen interface,

The user interest data may include one or more display menus. The display menu includes one or more selectable options. For example, the user interest data may include product data. The product data may include product advertisements and product promotions. The display menu may provide the user with the selectable option of viewing product promotional data associated with a displayed product advertisement. The display menu may provide the user with the option of selecting a particular product from a list of different products and view the associated product data.

The controller 402 continuously monitors the touch screen to determine whether a user has requested a display menu at step 708. If a user has not requested that a display menu be displayed on the display screen 408, the controller 402 continues to display the selected user interest data in accordance with the user interest data instructions as indicated in step 706. If the controller 402 determines that a user has requested a display menu, the display menu is retrieved from the local memory 404 and is displayed on the display screen 408 at step 710. In one embodiment, there is a plurality of display menus available for selection by a user. In another embodiment, the display menus are organized in a hierarchical manner.

At step 712, the controller 402 determines whether the user has selected an option from the display menu. If the user does not select an option from the display menu within a predetermined period of time, the controller 402 resumes the display of the pre-defined user interest data in accordance with the associated user interest data instructions as indicated in step 706. If the controller 402 determines that the user has selected a particular option from the display menu, the controller 402 displays the user interest data associated with the selected option at step 714.

The user is provided with the option of printing selected user interest data. In one embodiment, the user is provided with the option of printing the user interest data displayed on the display screen 408. In another embodiment, the user is provided with the option of printing additional information associated with displayed user interest data. For example, if the displayed user interest data consists of an advertisement for allergy medication, the user may be provided with the option of printing the side effects associated with taking the allergy medication. The user may also be provided with the option of printing coupons or rebates offers associated with the allergy medication.

The controller 402 monitors the touch screen to determine if the user has requested a print out of any user interest data at step 716. If the controller 402 determines that the user has requested a print out of selected user interest data, the controller 402 directs the printer 416 to print the requested user interest data at step 718. The method then proceeds to step 720. If the controller 402 determines that the user has not requested a print out, the method proceeds directly to step 720.

In one embodiment, the display menu includes options for selecting one or more types of forms. For example, the display menu may provide the user with the option of filling out a customer satisfaction survey for a product, a sweepstake entry form, or a request for additional product data associated with an advertised product from a product retailer or product manufacturer.

At step 720, the controller 402 determines whether the user has selected an option that requires the user to provide user supplied data, such as for example, user contact data, or user marketing data. If the user has selected such an option, in one embodiment, the controller 402 displays the requested form on the display screen 408 and the user provides the user supplied data via the touch screen display. In another embodiment, the controller 402 provides the user with prompts to provide the requested user supplied data. The controller 402 stores the user supplied data at step 722 and then returns to displaying the display menu at step 710. If the controller determines that the user has not selected an option that requires the user to provide user supplied data, the display screen returns to displaying the display menu on the display screen at indicated in step 710.

While a particular sequence of steps have been described above, it should be noted that a method 700 having substantially similar steps or steps in a different sequence are also considered to be within the scope of the invention. Also if any of the display menus or selectable displays remain dormant for a pre-defined period of time, or in other words no further user input is provided via the user interface for a pre-defined period of time, the controller 402 resumes the display of selected user interest data on the display screen 408 in accordance with the associated user interest data instructions.

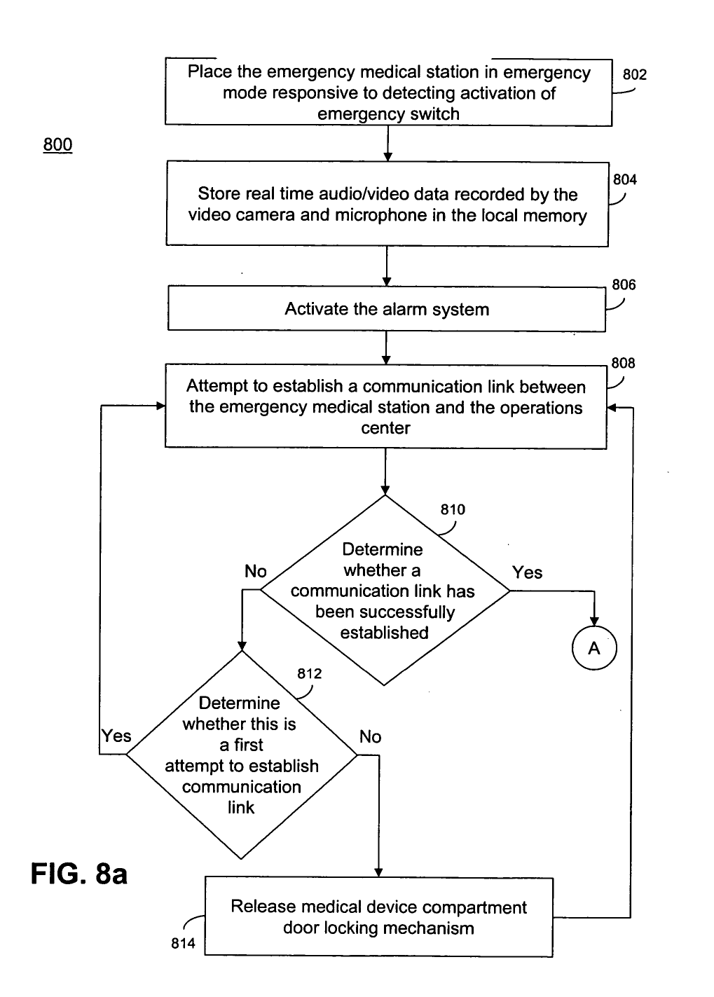

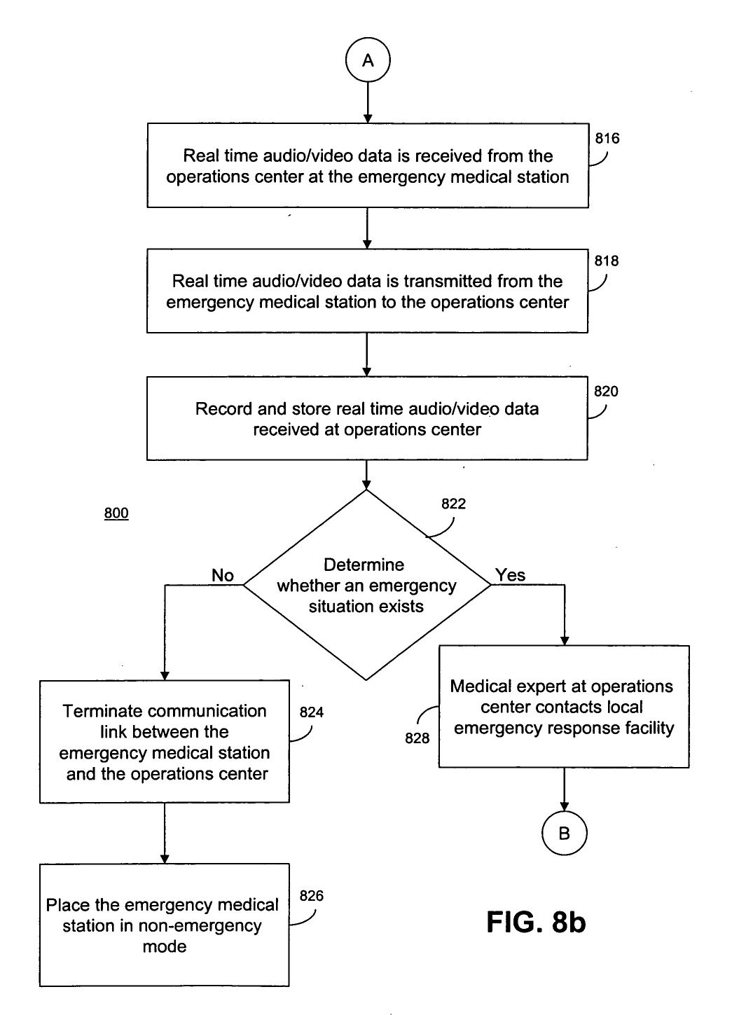

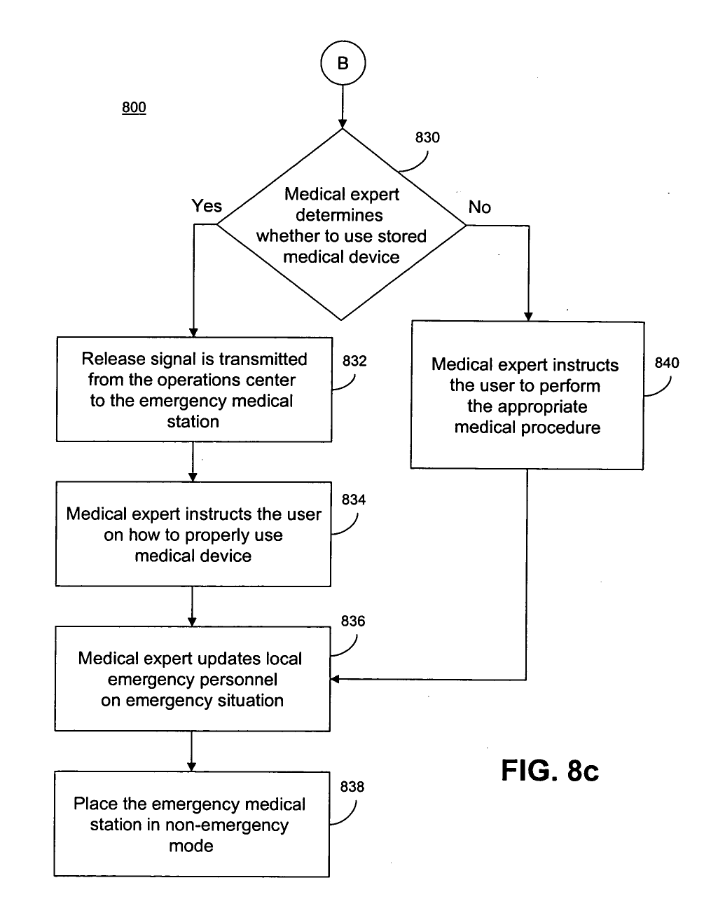

Referring to FIG. 8a-c, a flowchart illustrating a presently preferred embodiment of a method of responding to an emergency situation 800 in accordance with the principles of the present invention is shown. The method 800 begins at 802 with the controller 402 placing the emergency medical station 102 in emergency mode responsive to detecting that the emergency switch 420 has been activated. The emergency switch 420 generates an emergency signal upon activation. The controller 402 detects that the emergency switch 420 has been activated when the controller 402 detects the emergency signal.

The controller 402 immediately activates the video camera 410 and the microphone 412. At step 804, the real time audio/video data captured by the microphone 412 and the video camera 410 is stored in the local memory 404. In this manner, the emergency medical station 102 maintains an audio/video record of the emergency situation.

The controller 402 activates the alarm system 418 at step 806. In one embodiment, the alarm system 418 consists of a red strobe light and a siren. The red strobe light and the siren are activated for a predetermined period of time. In one embodiment, the predetermined period of time is approximately ten seconds.

The controller 402 attempts to establish a two way real time audio/video communication link between the emergency medical station 102 and the operations center 104 via the satellite communication system at step 808. The controller 402 determines whether the communication link was successfully established at step 810. If the controller 402 is unable to establish a communication link between the emergency medical system 102 and the operations center 104 within a predetermined period of time, the controller 402 determines whether this is the first attempt to establish the communication link at step 812.

If the controller 402 determines that this was the first attempt to establish a communication link between the emergency medical station 102 and the operations center 106 at step 812, the controller 402 waits for a predetermined period of time and then unlocks the medical device compartment door 508 locking mechanism 422 at step 814 and the user is provided with access to the medical device 506. The controller 402 continues attempts to establish a communication link between the emergency medical station 102 and the operations center 106 as indicated in step 808. If the controller 402 determines that this was not a first attempt to establish a communication link between the emergency medical station 102 and the operations center 106 at step 812, the controller 402 returns to step 808 and repeats attempts to establish the communication link.

If the controller 402 determines that the communication link has been successfully established between the emergency medical station 102 and the operations center 104 at step 810, the method proceeds to step 816.

At step 816, real time audio/video data is received from the operations center 106 at the emergency medical station 102. Real time live audio/video of a medical expert at medical expert station 306 is displayed on the display screen 408 at the emergency medical station 102. The real time live audio/video data is picked up by the microphone 314 and the video camera 310 located at the medical expert station 306.

The operations controller 302 directs the real time audio/video data to the data modem 304 for transmission to the emergency medical station 102 via the satellite communication system. The data modem 304 transmits the real time audio/video data to the communication satellite 108 via the router 118 and the satellite dish 120. The communication satellite 108 transmits the real time audio/video data to the emergency medical system 102 via the satellite dish 112 and the router 110.

The real time video data received from the operations center 106 is displayed on the display screen 408 and the received real time audio data is transmitted via the speakers 414. The user at the emergency medical station 102 can hear and see the medical expert at the operations center 106 on a real time basis.

The controller 402 transmits real time audio/video data from the emergency medical station 102 to the operations center 106 via the satellite communication system at step 818. The real time live audio/video data is picked up by the microphone 412 and the video camera 410 located at the emergency medical station 102.

The controller 402 directs the real time audio/video data to the communication module 406 for transmission to the operations center 106 via the satellite communication system. The communication module 406 transmits the real time audio/video data to the communication satellite 108 via the router 110 and the satellite dish 112. The communication satellite 108 transmits the real time audio/video data to the operations center 106 via the satellite dish 120 and the router 118.

The real time video data received from the emergency medical station 102 is displayed on the display screen 312 and the received real time audio data is transmitted via the speakers 316 at the operations center 106. The medical expert at the medical expert station 306 can hear and see the user and the patient on a real time basis. The real time two-way audio/video communications between the medical expert at the operations center 106 and the user at the emergency medical station 102 are recorded and stored in the operations center database 305 at step 820.

The real time two-way audio/video communication link permits the medical expert at an operations center 106 communicate directly with a user located at the medical emergency station 102. This permits the medical expert to evaluate the emergency situation at the emergency medical station 102 to determine whether an emergency situation exists at step 822.

If the medical expert determines that the situation at the emergency medical station 102 is not an emergency situation based on two way real time audio/video data exchanged between the emergency medical station 102, the medical expert authorizes the transmission of the emergency resolved signal from the operations center 106 to the emergency medical station 102. The controller 402 terminates the communication link between the emergency medical station 102 and the operations center 104 at step 824. The controller 402 places the emergency medical station 102 in non-emergency mode at step 826.

If the medical expert determines that the situation at the emergency medical station 102 is indeed an emergency situation based on two way real time audio/video communication with the user and/or person in distress at step 822, the medical expert contacts a local emergency response facility at step 828. Such emergency response facilities may include, but are not limited to, a fire department, a police department, and a local hospital. In one embodiment, the emergency medical station location data is stored in the local memory 404. The medical expert retrieves the location data from the emergency medical station 102 and informs the local emergency response facility of the location of the emergency situation. In another embodiment, the emergency medical station 102 includes a GPS unit. The medical expert obtains the emergency medical station location data from the GPS unit.

At step 830, the medical expert determines whether use of the medical device 506 stored in the locked medical device compartment 504 is necessary to treat the person in distress. The medical expert makes this determination based on real time two-way audio/video communication with the user at the emergency medical station 102 and/or the person in distress. For example, the medical expert may ask the user questions and to provide information regarding the physical condition of the person in distress. If for example, the medical expert determines that the person in distress is experiencing sudden cardiac arrest and the stored medical device 506 is a portable AED, the medical expert may determine that the use of the AED is necessary to save the distressed person's life.

If the medical expert determines that use of the stored medical device 506 is warranted, the medical expert directs the transmission of a release signal from the operations center 106 to the emergency medical station 102 at step 832. The controller 402 unlocks the locking mechanism 422 responsive to the received release signal. In one embodiment, the locking mechanism 422 is a servo driven locking mechanism. The controller 402 issues the appropriate commands to drive the servo to unlock the compartment door 508. Once the locking mechanism 422 is unlocked, the user can retrieve the medical device 506 stored in the medical device compartment 504. The medical expert instructs the user on the proper use of the medical device 506 via real time two-way audio/video communication link at step 834.

The medical expert continues to guide the user with assisting the person in distress until the local emergency response facility personnel arrive at the location of the emergency situation. At step 836, the medical expert updates the local emergency response facility personnel on the status of the emergency situation. The medical expert may update the local emergency facility of the patient's medical condition, a description of one or more medical treatments and the patient's response to the medical treatments.

Once the local emergency response facility personnel have gained control over the emergency situation, the medical expert authorizes the operations center 106 to transmit the emergency resolved signal to the emergency medical station 102. The controller 402 responds to the received emergency resolved signal by placing the emergency medical station 102 in non-emergency mode at step 838.

If the medical expert determines that use of the stored medical device 506 is not warranted at step 830, the medical expert does not authorize the transmission of a release signal from the operations center 106 and the medical device compartment 504 remains locked. At step 840, the medical expert remains on the two way audio/video communication link to guide the user through the appropriate medical procedures to assist the person in distress at step 526. For example, if the person in distress is bleeding, the medical expert may guide the user through CPR use of pressure bandages. The medical expert remains in two way real time audio/video communication with the user until personnel from the local emergency response facility arrive at the scene of the emergency. The method proceeds to step 836 where the medical expert updates the local emergency response facility personnel on the status of the emergency situation. The medical expert may update the local emergency response facility personnel of the patient's medical condition, a description of one or more medical treatments and the patient's response to the medical treatments.

Once the local emergency response facility personnel have gained control over the emergency situation, the medical expert authorizes the operations center 106 to transmit the emergency resolved signal to the emergency medical station 102. The controller 402 responds to the received emergency resolved signal by placing the emergency medical station 102 in non-emergency mode at step 838.

While a particular sequence of steps have been described above, it should be noted that a method 800 having substantially similar steps or steps in a different sequence are also considered to be within the scope of the invention.

The content distribution center 104 is operated by the emergency medical station 102 providers. As mentioned previously, the user interest data may include product data. Potential advertisers, such as for example, product manufactures, product retailers and service providers, wishing to promote their products and/or services can work with a content distribution advisor to coordinate the creation and/or display of their product data on one or more emergency medical stations 102. In one preferred embodiment, compensation is received from the advertisers in exchange for promoting their products and/or services on one or more emergency medical stations 102. The compensation received from the advertisers is used to find the costs associated with the manufacture, placement, operation, insurance premiums, and training associated with the placement of emergency medical stations 102 in a one or more locations. Other types of costs beyond those described may be covered by the compensation received from advertisers without departing from the spirit of the invention, such as decreased costs and/or increased revenue resulting from promotion of the goods and/or services of the entity that owns or leases the facility that houses the emergency medical station.

For example, an airport manager may wish to place one or more emergency medical stations 102 in an airport. The airport management contacts the emergency medical system provider according to the present invention and makes a request for the placement of one or more emergency medical stations 102 in the airport. The emergency medical station provider supplies the requested emergency medical stations to the airport at a given charge, at a reduced charge or free of charge. The emergency medical system provider may also provide coverage of insurance costs associated with the placement of the emergency medical station, and may also provide training costs associated with training selected airport personnel to operate the medical device stored in the emergency medical station 102. One or more of the costs described above are covered by the compensation received from advertisers for displaying user interest data associated with the advertisers' product and/or services on one or more emergency medical stations 102 or are covered by an overall reduction in the insurance rates for the related facility housing the emergency medical stations 102.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

1. An emergency medical station comprising:
a controller;
a video camera communicatively coupled to the controller;
a communication module communicatively coupled to the controller, the communication module being operable to transmit real time video data from the video camera to an operations center, to receive real time video data from the operations center and to receive a release signal from the operations center;
a display screen communicatively coupled to the controller, the display screen being operable to display the received real time video data; and
a medical device compartment with a locking mechanism communicatively coupled to the controller, the locking mechanism being operable to unlock the medical device compartment responsive to the release signal.

2. The emergency medical station of claim 1, further comprising an emergency switch communicatively coupled to the controller and operable to generate an emergency signal responsive to activation.

3. The emergency medical station of claim 2, further comprising an alarm system communicatively coupled to the controller and operable to generate an alarm responsive to the emergency signal.

4. The emergency medical station of claim 3, wherein the alarm system comprises an alarm selected from the group consisting of a strobe light and a siren.

5. The emergency medical station of claim 2, wherein the controller establishes a communication link between the emergency medical station and the operations center responsive to the emergency signal.

6. The emergency medical station of claim 5, wherein the communication link is established via a satellite communication network.

7. The emergency medical station of claim 5, wherein the controller unlocks the locking mechanism after a predefined period of time following a failure to establish a communication link.

8. The emergency medical station of claim 1, wherein the locking mechanism is unlocked responsive to a disruption of power supplied to the emergency medical station.

9. The emergency medical station of claim 1, wherein the locking mechanism comprises a servo driven locking mechanism.

10. The emergency medical station of claim 1, wherein the display screen consists of a display screen selected from the group consisting a touch screen display, a liquid crystal display and a cathode ray tube display.

11. The emergency medical station of claim 1, further comprising:
a microphone communicatively coupled to the communication module, wherein the communication module is operable to transmit real time audio data received at the microphone to the operations center; and
a speaker communicatively coupled to the communication module, wherein the communication module is operable to transmit real time audio data received from the operations center.

12. The emergency medical station of claim 11, further including a local memory communicatively coupled to the controller and operable to store the real time audio data received at the microphone and the real time audio data received from the operations center.

13. The emergency medical station of claim 11, wherein the operations center stores the real time audio data received from the emergency medical station and the real time audio data transmitted to the emergency medical station in an operations center database.

14. The emergency medical station of claim 1, further comprising a standalone automatic external defibrillator unit for storage in the medical device compartment.

15. The emergency medical station of claim 1, wherein the real time video data received from the operations center comprises real time video of a medical expert.

16. The emergency medical station of claim 1, further including a back up power source operable to power the emergency medical station responsive to a failure of a primary power source.

17. The emergency medical station of claim 1, further including a local memory communicatively coupled to the controller and operable to store the real time video data recorded by the video camera and the real time video data received from the operations center.

18. The emergency medical station of claim 1, wherein the operations center stores the real time video data received from the emergency medical station and the real time video transmitted to the emergency medical station in an operations center database.

19. A method of responding to an emergency situation, the method comprising:
establishing a communication link between an emergency medical station and an operations center;
transmitting real time video data from the emergency medical station to the operations center;
receiving real time video data from the operations center at the emergency medical station;
determining whether a medical device compartment locking mechanism should be unlocked based on the real time video data exchange between the emergency medical station and the operations center; and
unlocking the locking mechanism based on the determination.

20. The method of claim 19, further comprising placing the emergency medical station one of a non-emergency mode and an emergency mode wherein the emergency medical station is placed in an emergency mode responsive to activation of an emergency switch.

21. The method of claim 20, wherein the step of establishing the communication link comprises establishing a communication link in emergency mode.

22. The method of claim 20, further comprising activating an alarm system in emergency mode.

23. The method of claim 19, wherein the step of establishing the communication link between the emergency medical station and the operations center comprises establishing a communication link between the emergency medical station and the operations center via a satellite communication network.

24. The method of claim 19, further comprising unlocking the locking mechanism after a predefined period following a failure to establish a communication link between the emergency medical station and the operations center.

25. The method of claim 19 further comprising unlocking the locking mechanism responsive to disruption of power supplied to the emergency medical station.

26. The method of claim 19, further comprising storing the real time video data transmitted to the operations center and the real time video data received from the operations center in a local memory.

27. The method of claim 19, further comprising storing the real time video data transmitted to the emergency medical station and the real time video data received from the emergency medical station in an operations database.

28. The method of claim 19, further comprising:
transmitting real time audio data from the emergency medical station to the operations center; and
receiving real time audio data from the operations center at the emergency medical station.

29. The method of claim 28, further comprising storing the real time audio data transmitted to the operations center and the real time audio data received from the operations center in a local memory.

30. The method of claim 28, further comprising storing the real time audio data transmitted to the emergency medical station and the real time audio data received from the emergency medical station in an operations database.

31. The method of claim 19, further comprising:
determining whether to contact a local emergency response facility based on the exchange of real time video data between the emergency medical station and the operations center; and
contacting the local emergency response facility based on the determination.

32. A computer readable medium storing a computer program for responding to an emergency situation, the computer program comprising:
computer readable code for establishing a communication link between an emergency medical station and an operations center;
computer readable code for transmitting real time video data from the emergency medical station to the operations center;
computer readable code for receiving real time video data from the operations center at the emergency medical station; and
computer readable code unlocking a locking mechanism of a medical device compartment responsive to a release signal received from the operations center wherein, the release signal is generated from the operations center based on an assessment of the real time video data exchanged between emergency medical station and the operations center.

33. The computer readable medium of claim 32, further comprising computer readable code for placing the emergency medical station in one of a non-emergency mode and an emergency mode wherein, the emergency medical station is placed in emergency mode responsive to an emergency signal.

34. The computer readable medium of claim 33, wherein the computer readable code for establishing the communication link comprises computer readable code for establishing a communication link responsive to the emergency signal.

35. The computer readable medium of claim 33, further comprising computer readable code for activating an alarm system responsive to the emergency signal.

36. The computer readable medium of claim 33, further comprising:
computer readable code for storing the real time video data recorded by the video camera in a local memory responsive the emergency signal; and
computer readable code for storing the real time video data received from the operations center responsive to the emergency signal.

37. The computer readable medium of claim 32, wherein the computer readable code for establishing the communication link between the emergency medical station and the operations center comprises computer readable code for establishing a communication link via a satellite communication network.

38. The computer readable medium of claim 32, further comprising computer readable code for unlocking the locking mechanism after a predefined period of time following a failure to establish a communication link between the emergency medical station and the operations center.

39. The computer readable medium of claim 32, further comprising computer readable code for unlocking the locking mechanism responsive to disruption of power supplied to the emergency medical station.

40. The computer readable medium of claim 33, further comprising:
computer readable code for transmitting real time audio data from the emergency medical station to the operations center responsive to the emergency signal; and
computer readable code for receiving real time audio data from the operations center at the emergency medical station responsive to the emergency signal.

41. The computer readable medium of claim 40, further comprising:
computer readable code for storing the real time audio data received by the microphone in a local memory responsive to the emergency signal; and
computer readable code for storing the real time audio data received from the operations center responsive to the emergency signal.

42. An emergency medical station comprising:
a controller operable to place the emergency medical station in one of a non-emergency mode and an emergency mode;
a video camera communicatively coupled to the controller;
a communication module communicatively coupled to the controller, the communication module being operable to transmit real time video data from the video camera to an operations center in emergency mode and to receive real time video data from the operations center in emergency mode; and
a display screen communicatively coupled to the controller and operable to display user interest data in non-emergency mode and operable to display the real time video data received from the operations center in emergency mode.

43. The emergency medical station of claim 42, further comprising an emergency switch wherein, the controller is operable to place the emergency medical station in emergency mode responsive to activation of the emergency switch.

44. The emergency medical station of claim 42, wherein the non-emergency mode is a default operating mode.

45. The emergency medical station of claim 42, wherein the communication module is operable to receive the user interest data from a content distribution center.

46. The emergency medical station of claim 42, further including a local memory communicatively coupled to the controller and operable to store the user interest data.

47. The emergency medical station of claim 42, further comprising a user interface communicatively coupled to the controller.

48. The emergency medical station of claim 47, wherein the user interface consists of a user interface selected from the group consisting of a touch screen display, a card reader, a bar code reader, and a pointer control.

49. The emergency medical station of claim 47, wherein the user interest data includes selectable user interest data that can be selectively displayed on the display screen responsive to a user input received via the user interface.

50. The emergency medical station of claim 47 further comprising a local memory operable to store user supplied data received via the user interface.

51. The emergency medical station of claim 50, wherein the communication module is operable to transmit the stored user supplied data to the content distribution center.

52. The emergency medical station of claim 47, further including a printer operable to print user interest data selected by a user via the user interface.

53. The emergency medical station of claim 42, wherein the controller is operable to display the user interest data on the display screen in a pre-defined sequence.

54. The emergency medical station of claim 42, wherein the user interest data comprises product data.

55. A method of responding to an emergency situation, the method comprising:
providing an emergency medical station having a default non-emergency mode wherein user interest data is displayed on a display screen in non-emergency mode;
placing the emergency medical station in emergency mode responsive to activation of an emergency switch;
transmitting real time video data from the emergency medical station to the operations center in emergency mode;
receiving real time video data from an operations center at the emergency medical station in emergency mode; and
displaying the real time video received from the operations center on the display screen in emergency mode.

56. The method of claim 55, further comprising receiving user interest data from a content distribution center.

57. The method of claim 56, further comprising storing the received user interest data in a local memory.

58. The method of claim 55, wherein displaying the user interest data further comprises displaying the user interest data in a pre-defined sequence.

59. The method of claim 55, further comprising providing a user interface for interfacing with the user interest data.

60. The method of claim 59, further comprising selecting user interest data for display on the display screen via the user interface.

61. The method of claim 59, further comprising selecting user interest data for printing on a printer via the user interface.

62. The method of claim 55, further comprising storing user supplied data received via the user interface in a local memory.

63. The method of claim 62, further comprising transmitting the stored user supplied data to a content distribution center.

64. A computer readable medium storing a computer program for responding to an emergency, the computer program comprising:
computer readable code for placing an emergency medical station in a default non-emergency mode and for placing the emergency medical station in emergency mode responsive to an emergency signal;
computer readable code for displaying user interest data on a display screen in non-emergency mode;
computer readable code for transmitting real time video data from the emergency medical station to the operations center in emergency mode;
computer readable code for receiving real time video data from the operations center at the emergency medical station in emergency mode; and
computer readable code for displaying the real time video data received from the operations center on the display screen in emergency mode.

65. The computer readable medium of claim 64, further comprising computer readable code for receiving the user interest data from a content distribution center.

66. The computer readable medium of claim 65, further comprising computer readable code for storing the received user interest data in a local memory at the emergency medical station.

67. The computer readable medium of claim 64, wherein the computer readable code for displaying the user interest data further comprises computer readable code for displaying the user interest data in a pre-defined sequence.

68. The computer readable medium of claim 64, further comprising computer readable code for displaying user interest data selected via a user interface on the display screen.

69. The computer readable medium of claim 64, further comprising computer readable code for accepting user input pertaining to user interest data via a user interface.

70. The computer readable medium of claim 64, further comprising computer readable code for printing user interest data selected via a user interface.

71. The computer readable medium of claim 64, further comprising computer readable code for storing user supplied data received via a user interface in a local memory.

72. The computer readable medium of claim 71, further comprising computer readable code for transmitting the stored user supplied data to a content distribution center.
